(19) United States
(12) Patent Application Publication
(10) Pub. No.: US 2003/0125617 A1
(43) Pub. Date: Jul. 3, 2003

(54) Method of magnetic resonance imaging

(76) Inventors: Atle Bjornerud, Oslo (NO); Lars Johansson, Uppsala (SE)

Correspondence Address: KENYON & KENYON, ONE BROADWAY, NEW YORK, NY, 10004, US

(21) Appl. No.: 10/259,156

(22) Filed: Sep. 27, 2002

Related U.S. Application Data
(63) Continuation of application No. PCT/NO01/00136, filed on Mar. 30, 2001.
(60) Provisional application No. 60/210,910, filed on Jun. 12, 2000.

(30) Foreign Application Priority Data
Mar. 31, 2000 (GB) 0007873.3

(51) Int. Cl. A61B 5/05

(52) U.S. Cl. 600/411

(57) ABSTRACT

A method of MR imaging to monitor the status of any chronic and/or acute inflammatory disease, in particular to the use of MR imaging to monitor and assess the inflammatory response to organ transplants wherein a blood-pool MR contrast agent composition is administered.

This invention relates to improvements in and relating to magnetic resonance (MR) imaging, to the use of MR imaging to monitor the status of any chronic and/or acute inflammatory disease, in particular to the use of MR imaging to monitor and assess the inflammatory response to organ transplants.

To identify and localise inflammatory foci is a critical step in appropriate treatment of patients with or suspected of having inflammatory processes, e.g. in immunocompromised patients. According to the present invention, we now propose that inflammatory foci may be visualised due to the locally enhanced vascular permeability.

Viewed from one aspect the invention provides a method of monitoring inter-endothelial discontinuities caused by an inflammatory disease in a human or vascularized animal subject, which comprises administering into the vasculature of a said subject a blood-pool MR contrast agent composition, and monitoring the MR signal for an increase in enhancement in said inter-endothelial discontinuities, (e.g. at a time of at least 5 minutes) following administration of said contrast agent.

In organ transplant surgery, a major problem is organ rejection due to the inflammatory response to organ transplants. As the transplanted organ is within the patient's body, onset of rejection is not generally visibly apparent to the physician and rejection can be life-threatening if it is not detected before organ failure has reached an advanced stage. There is thus a need for a non-invasive technique for monitoring the transplanted organ for signs of rejection.

We now propose the use of contrast-enhanced MR imaging for monitoring inter-endothelial discontinuities caused by an inflammatory disease in a human or vascularized animal subject, especially monitoring organ transplants for signs of rejection. More particularly we propose the use of vascularly administered blood-pool MR contrast agents, especially magnetic metal oxides, more especially particulate supermagnetic metal oxides, e.g. ultra small superparamagnetic iron oxides (USPIOs), in this regard.

Viewed from a further aspect the invention provides a method of monitoring a transplanted organ in a human or vascularized animal (e.g. mammalian, avian or reptilian) subject, which comprises administering into the vasculature of a said subject into which an organ has been transplanted a blood-pool MR contrast agent composition, and monitoring the MR signal from the transplanted organ for an increase in enhancement, e.g. at a time of at least 5 minutes (e.g. 5 to at least 100 minutes, preferably 15 to 60 minutes) following administration of said composition.

Viewed from a yet further aspect the invention provides a method of detecting an indication of transplanted organ rejection which method comprises monitoring the MR signal from a transplanted organ in a vascularised human or non-human (e.g. mammalian) subject, following administration into the vasculature thereof of a blood-pool MR contrast agent, for a signal enhancement which is increased relative to that of the blood or of a non-transplanted vascularized tissue or organ, or which increases with time following the first pass of the agent through the transplant organ.

Viewed from a yet further aspect the invention provides a method of generating enhanced images of a human or a vascularized animal body previously administered with a blood-pool MR contrast agent composition, which method comprises generating an MR image of at least part of the inter-endothelial discontinuities of said body.

In the methods of the invention, a blood-pool MR contrast agent composition is administered into the vasculature of the organ-transplant patient. By a blood pool MR contrast agent is meant a magnetic (e.g. paramagnetic, ferromagnetic, ferrimagnetic or superparamagnetic) material, preferably a magnetic iron oxide, capable of reducing the T₁ and/or T₂/T₂* of water protons and which if administered into the vascular space does not significantly leak out into the interstitium during the time course of the imaging procedure, i.e. it is essentially confined to the vascular space until excreted or metabolized. Examples of formulations of such blood pool agents include polymeric chelates (e.g. cascade polymers or dendrimers carrying metallated chelate groups) and particulates, in particular magnetic metal oxides and liposomes. Generally the agent should have a blood half life of at least 5 minutes, preferably at least 30 minutes. By way of contrast, the first parenteral MR contrast agents Gd DTPA (Magnevist® from Schering), Gd DTPA-bismethylamide (Omniscan® from Nycomed Amersham) and Gd HP-D03A (ProHance®) are all extracellular fluid MR agents; they are water-soluble mono-chelates which following administration into the vasculature rapidly extravasate into the interstitium.

Viewed from a further aspect the invention provides the use of a blood-pool MR contrast agent, preferably an ultra small superparamagnetic metal oxide, for the manufacture of a contrast agent composition for use in a method of diagnosis which involves MR monitoring the status of any chronic and/or acute inflammatory disease in a human or vascularized animal subject, preferably by generation of a magnetometric image of at least part of the human or non-hunam body, preferably of a transplanted organ subject to detect indications of organ rejection.

Viewed from a yet further aspect the invention provides the use of a blood-pool contrast agent composition, preferably comprising an ultra small superparamagnetic metal oxide, in MR imaging of at least part of a human or vascularized animal subject for monitoring inter-endothelial discontinuities caused by an inflammatory disease by monitoring the MR signal for an increase in enhancement in said inter-endothelial discontinuities following administration of said composition.

Blood pool agents of particular use in the method of this invention include low molecular weight chelates which bind to blood proteins, e.g. blood proteins such as albumin, for example DTPA or DOTA derivatised with protein binding groups, e.g. lipophilic side chains such as aromatic moieties, e.g. one or more phenyl ring systems. One such example is MS-325/Angiomark of EPIX.

Suitable polymer based contrast agents for use in the method of the present invention can be carbohydrate or protein based, e.g. CMD-DTPA-Gd of Guerbet (Carboxymethyl dextran-GddTPA conjugates), GdDTPA polylysine conjugates, or cascade or dendrimer polymers, e.g. Gadomer 17 of Schering AG or similar cascade polymers as described in U.S. Pat. No. 5,874,061 (of Schering AG), herein incorporated by reference.

Suitable iron oxide (or doped iron oxide) based contrast agents for use in the method of the present invention are known in the field under the name of SPIO (superparamagnetic iron oxides) or USPIO (ultrasmall superparamagnetic iron oxides). Examples include carbohydrate stabilised iron oxide particles, e.g. dextran-stabilised particles such as Combidex of Advanced Magnetics, and the degraded starch coated USPIOs. One such agent is known as NC100150 a blood pool contrast agent coated with a starch residue and further coated with a PEG component (Clariscan™, Nycomed Amersham), e.g. disclosed in WO 97/25073 (see Ex. 12).

More particularly the magnetic iron oxide contrast agent is preferably a water-dispersible material comprising magnetic iron oxide particles having on their surfaces (e.g. as a coating), an optionally modified carbohydrate or polysaccharide or derivative thereof, e.g. a glucose unit containing optionally modified polysaccharide or derivative thereof, preferably an optionally modified dextran or starch or derivative thereof, for example a cleaved (e.g. oxidatively cleaved) starch or carboxylated dextran. Such iron oxide complexes preferably also comprise a further material (e.g. coating material), especially one which inhibits opsonization, e.g. a hydrophilic polymer, preferably a functionalized polyalkylene oxide, more preferably a functionalized polyethylene glycol (PEG), in particular methoxy PEG phosphate (MPP).

The iron oxide complexes preferably have a core (i.e. iron oxide particle) diameter (mode diameter) of 1 to 15 nm, more preferably 2-10 nm, especially 3-7 nm, a total diameter (mode particle size) of 1 to 100 nm, more preferably 5-50 nm, especially preferably 10-25 nm, an r₂/r₁ ratio at 0.47T and 40° C. of less than 3, more preferably less than 2.3, still more preferably less than 2.0, especially preferably less than 1.8. The saturation magentization (Msat) at 1T is preferably 10 to 100 emu/gFe, more preferably 30-90 emu/gFe.

Other particulate based systems of use in the method of the present invention include liposomal or emulsion based agents.

Furthermore, compound 7228 of Advanced Magnetics can be used in the method of the present invention, as can the materials described in WO 91/12025, WO 90/01899, WO 88/00060, WO 91/12526 and WO 95/05669, all to Advanced Magnetics, and those described in WO92/11037 and WO90/01295, all of which publications are incorporated herein by reference.

Preferably, the blood-pool agent will function as a positive or T₁ agent, i.e. increasing the MR signal from the regions into which it distributes. However negative or T₂ (or T₂*) agents which serve to reduce the MR signal may also be used. However the agent will especially preferably be a particulate superparamagnetic metal oxide, especially an ultra small superparamagnetic iron oxide (USPIO)

In the methods of the invention it is not necessary to generate an image of the transplanted organ; all that is required is to monitor the MR signal from a region of interest that contains some or all of the parenchyma of the transplanted organ. For this reason, by way of example, the delta signal intensity provides a particularly suitable parameter to monitor. Obviously however, the identification of the region of interest may involve generation of an image of the transplanted organ; however this may of course be done pre-contrast. Nevertheless it will generally be desirable to generate post-contrast images of the transplanted organ as in this way the location and spatial extent of inflammation indicative of rejection may be determined. For organs which contain large blood vessels or, like the heart, large blood-containing cavities, the region of interest should generally be chosen to minimise the fraction which corresponds to large blood vessel or blood (i.e. heart) chamber and to maximise the fraction which corresponds to organ parenchyma.

The method will generally involve selection of a comparison region of interest in order that the relative signal enhancement increase in rejected organ tissue may most readily be detected. For this purpose a healthy organ or healthy tissue, e.g. muscle, or a relatively large blood vessel may be selected. Likewise it will generally be desirable to monitor the MR signals at a plurality of times following contrast agent administration, e.g. over a period of up to 3 hours following administration, more generally over a period of at least one quarter of the blood half life of the contrast agent. In a particularly preferred embodiment, the temporal development of the MR signals from the regions of interest will be monitored (and preferably plotted) so as to determine whether the sign of d(S)/dt (where S is signal intensity) is different or of opposite polarity for the region of interest in the transplanted organ and the comparison region of interest. That is to say where (following first pass) signal intensity is increasing with time in the transplanted organ and (also following first pass) is decreasing in the corresponding comparison region, this may be taken as an indication that organ rejection is occurring. The maximum ds/dt value for that region of interest may moreover be used as a measure of severity of organ rejection.

The monitoring according to the invention is preferably made once the contrast agent has equilibrated in the blood-pool. The contrast agent is preferably administered in a bolus e.g. over a period of less than 3 minutes, preferably less than 100 seconds, more preferably less than 60 seconds, for example 0.3 to 10 seconds. Less preferably it may be administered by infusion, e.g. over a period of 3 to 10 minutes, preferably 3 to 6 minutes. Contrast medium administration rates will desirably be in the range 0.01 to 10 mL/sec, preferably 0.3 to 3 mL/sec. If desired a chaser, e.g. of physiological saline, may also be administered. The achievement of uniform distribution in the blood-pool may readily be determined by following the signal from the blood in a large blood vessel where the peaks in signal intensity following first and second pass flatten out, the contrast agent can be taken to be equilibrated throughout the blood-pool.

Administration of the contrast agent is preferably into a vein, e.g. a vein in a limb or, in the case of an animal, the tail.

In the methods of the invention, the MR contrast agent is preferably administered in a dose which gives a blood concentration of 0.01 to 10 mM, eg for USPIOs a dosage of 0.5 to 8 mg Fe/kg bodyweight, more preferably 1 to 6 mg Fe/kg, especially 2 to 5 mg Fe/kg.

The contrast agent may be formulated for use in the method of the invention with conventional pharmaceutical carriers and excipients. Typically they will be in aqueous dispersion form, e.g. at an iron content of 10 to 50 mg Fe/mL, preferably 20 to 40 mg Fe/mL. Excipients that may be present include pH modifiers, chelating agents, viscosity modifiers, osmolality modifiers, etc.

An example of a composition that may be used is an aqueous suspension of an USPIO blood pool MR contrast agent prepared according to the description in Example 12 of WO97/25073, eg a suspension with the following characteristics: [Fe]=30.2 mg Fe/mL; density 1.0589 g/mL; r₁=19.3 s⁻¹mM; r₂=31.2 s⁻¹mM; r₂/r₂=1.61 (at 20 MHz and 37° C.); saturation magnetization (Msat)=84 emu/g Fe.

The MR signal acquisition used in the methods of the invention will preferably be a T₁ weighted acquisition, e.g. a conventional spin echo or gradient echo sequence, generally with TE and TR of 1 to 10 ms and 2 to 50 ms respectively for a 1.5T magnet for gradient echo sequences and 5 to 40 ms and 100 to 1000 ms respectively for spin echo sequences. Two or three dimensional imaging procedures may be used and relatively low resolution may be sufficient unless the spatial extent of a localised inflammation in the transplanted organ is to be mapped.

The methods of the invention work since the blood-pool agent doesn't normally break through the blood vessel walls in the transplanted organ or healthy comparison organ or tissue and is gradually cleared from the blood, generally through the reticuloendothelial system for particulates (such as USPIOs) or the kidneys. In rejected organ tissue however an inflammatory response causes breakdown of the inter-endothelial gap junctions causing inter-endothelial discontinuities allowing the contrast agent to leak into the interstitial space.

Any chronic or acute inflammatory disease may be monitored by the present invention, e.g. asthma, atherosclerosis, inflamed joint (for example arthritis, periarthritis and polyarthritis), inflamed joint membrane (e.g. synovitis), inflamed kidney (e.g. nephritis, pyelitis, pyelonephritis and pypnephritis), inflamed liver (e.g. hepatitis), inflamed lungs (e.g. pneumonteritis, pneumonia, pneumonitis and pulminitis), inflamed marrow/bone (e.g. osteomyelitis), inflamed pancreas (e.g. pancreatitis), myocarditis, asthma and atherosclerosisin, in particular rheumatoid arthritis.

Documents referred to herein are hereby incorporated by reference.

The present invention will now be described further with reference to the following non-limiting Examples and the accompanying Figures in which:

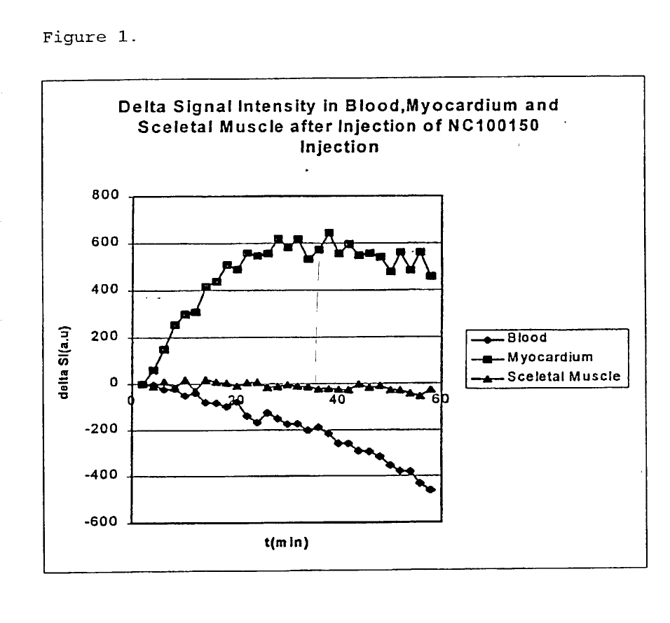

FIG. 1 is a plot of delta signal intensity against time for blood, myocardium and skeletal muscle following intravenous injection of an USPIO contrast agent in a rat with a transplanted heart.

An aqueous suspension of an USPIO blood pool MR contrast agent prepared according to the description in Example 12 of WO97/25073 was used in Example 1. The characteristics of this suspension were: [Fe]=30.2 mg Fe/mL; density 1.0589 g/mL; r₁=19.3 s⁻¹mM; r₂=31.2 s⁻¹mM⁻¹; r₂/r₁=1.61 (at 20 MHz and 37° C.); saturation magnetization (Msat)=84 emu/g Fe.

### EXAMPLE 1

2 mg Fe/ml bodyweight of the contrast medium was injected over a period of 5 sec into a femoral vein of a rat (250 g) which had previously received a heart transplant which was being rejected.

The rat was monitored over a period of 60 minutes in a 1.5T Philips MR imaging apparatus using a 3D spoiled gradient echo sequence with TR 20 ms, TE 3.1 ms and flip 35°. Normalised delta signal intensity values for regions of interest corresponding to (♦) blood, (▴) skeletal muscle and (▪) myocardium were detected and are plotted in FIG. 1 of the accompanying drawings. The signal in blood drops considerably over time as the contrast agent is cleared from the blood. Skeletal muscle also drops due to half life effects but to a lesser extent since the bulk of the signal is from tissue rather than blood in vessels in the muscle. The myocardium signal however increases significantly over about 30 minutes as contrast agent leaks into the interstitium before gradually dropping as a result of wash out of contrast agent.

### EXAMPLE 2

Animals

Male rats weighing 175-250 g were used for the experiments.

Heterotopic Heart Transplantation

Heterotopic heart transplantations were performed whit anastomosis of the aorta of the donor heart to the right common carotid artery of the recipient, and of the pulmonary artery to the right jugular vein, using a non-suture technique. In brief, the carotid artery and the jugular vein were dissected free, cross-clamped caudally, and cut cranially. Short plastic tubes were placed around the vessels which were then turned inside out over the tubes and fixed with ligatures. The donor heart was anastomosed by pulling the vessels of the graft over the tubes and fastening them with ligatures. When the transplantation was completed a single dose of cefuroxime, 20 mg/rat, was administered intramuscularly. Graft function was monitored by daily palpation.

Experimental Groups

Two repeated MRI investigations were performed in the same animal, 2 and 6 days after transplantation. Rats with allogeneic (n=6) and those with syngeneic (n=6) transplants were studied. In addition, three more rats with allogeneic transplants were included in the study. These rats were analysed on day 5 (n=2) and day 7 (n=1).

Histopathological Analysis

Immediately after the second MR imaging, the rat was sacrificed and the transplanted heart was excised and fixed in 4% formalin. Subsequently, the graft was embedded in paraffin, cut into 4 μm thick sections and stained with eosin and Mayer's hematoxylin. All slides were evaluated blindly with respect to general morphology, edema, infiltrating cells, and myocyte necrosis. The sections were scored arbitrarily according to an ascending five-step scale (1-5) where 1 represented grafts with mild interstitial edema and no or few infiltrating cells and 5 represented grafts with pronounced interstitial edema and massive infiltration.

MR Imaging

At MR imaging a scan with 21 dynamic phases was performed, with the first phase (time-point) 1 minute post injection and the last phase 45 minutes post injection. A 1.5 T clinical scanner with a 22 mm surface coil and the rat in a supine position was used for the acquisition. The acquisition sequence was a 3D spoiled gradient echo with a TR/TE of 20/3.1 ms and a flip angle of 35°. The FOV was 70 mm, with 256×256 matrix yielding an in-plane resolution of 0.27 mm and a slice thickness of 0.5 mm.

Contrast Agent

NC100150 Injection (Clariscan™, Nycomed Imaging AS), an ultrasmall superparamagnetic iron oxide (USPIO) contrast agent with a particle size of 15 nm, was injected into the femoral vein at a dose of 2 mg Fe/kg body weight. The dose was chosen to maximize the T1-relaxation effect while maintaining the T2*-relaxation effect at a level at which it can be neglected for the chosen scan parameters.

Statistical Analysis

The MR signal intensity was measured in the myocardium and blood using a region of interest containing at least 20 pixels. The relative change in signal intensity was calculated at each time point, using the first time point after injection as a reference in each animal. The syngeneic (n=6) and the allogeneic (n=6) groups were averaged separately and differences between the two groups were tested for statistical significance with a Student's t-test at each time point. In all animals, 6 with syngeneic and 9 with allogeneic transplants, the correlation between the histologically classified morphological changes and the mean of the relative signal intensity change at the last ten measurement points was tested using the Spearman rank order correlation coefficient. This correlation was also tested in the allogeneic group alone.

Results:

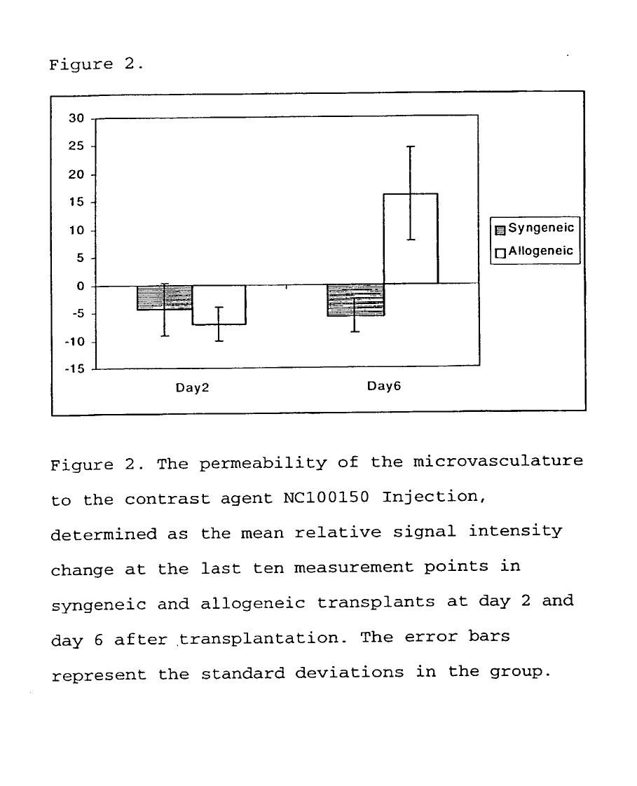

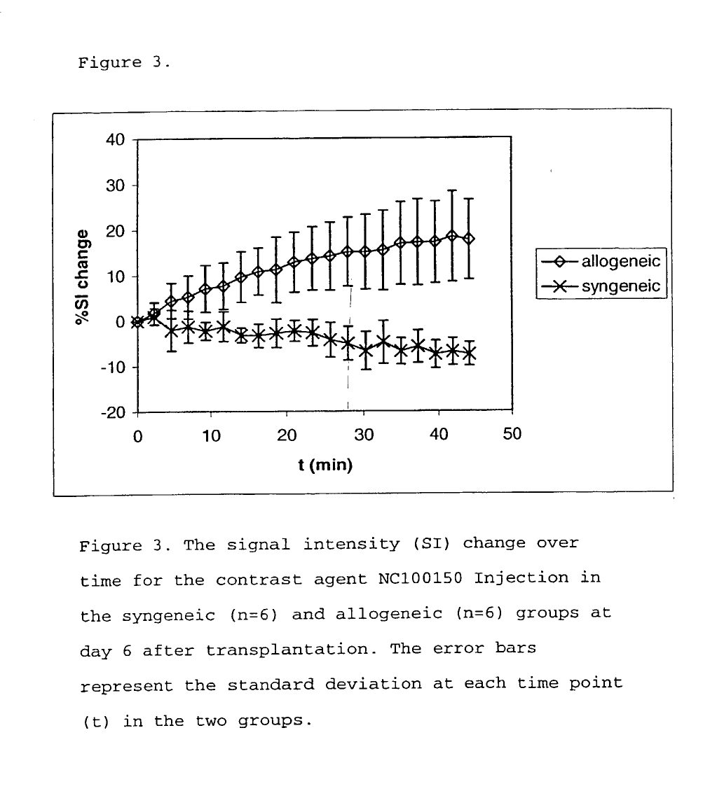

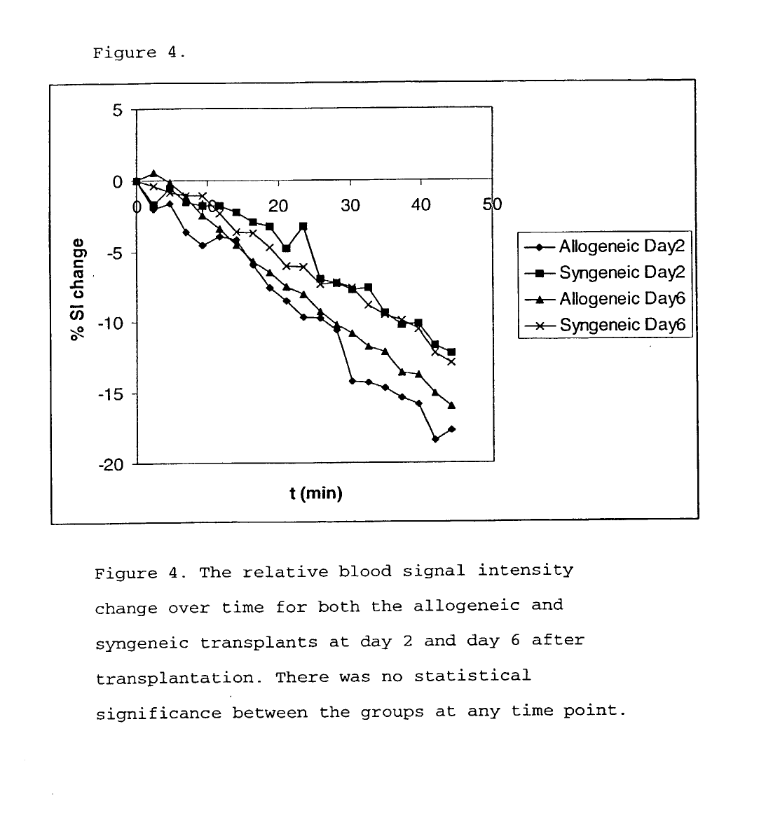

At day 2 after transplantation the relative signal intensity change in the myocardium did not differ significantly between the two groups (FIGS. 2 and 3). At day 6 however, the mean relative change in signal intensity at the last ten time points was significantly greater in the allogeneic than in the syngeneic grafts (+16.1 vs −5.74; p<0.0005). When determining the statistical significance of the difference between the two groups over time, it was found that the p value was lower than 0.01 14 minutes after injection and then decreased with time. The clearance of the contrast agent, measured as change in signal intensity in the blood did not differ between the syngeneic and allogeneic groups or between measurements performed on day 2 and day 6 (FIG. 4)

The cardiac grafts in the syngeneic group all displayed a homogeneous morphology, scored as grade 1, with mild interstitial edema and with cellular infiltration restricted to the peripheral region of the transplant. In the allogeneic group there was a wider variation, with scores ranging from 3 to 5. A positive correlation was found between the mean relative change in signal intensity at the last ten time points and the histologically assessed rejection grade both on analysis of the allogeneic group alone (r=0.89; p<0.005), and when both the syngeneic and the allogeneic groups were included (r=0.94; p<0.0005).

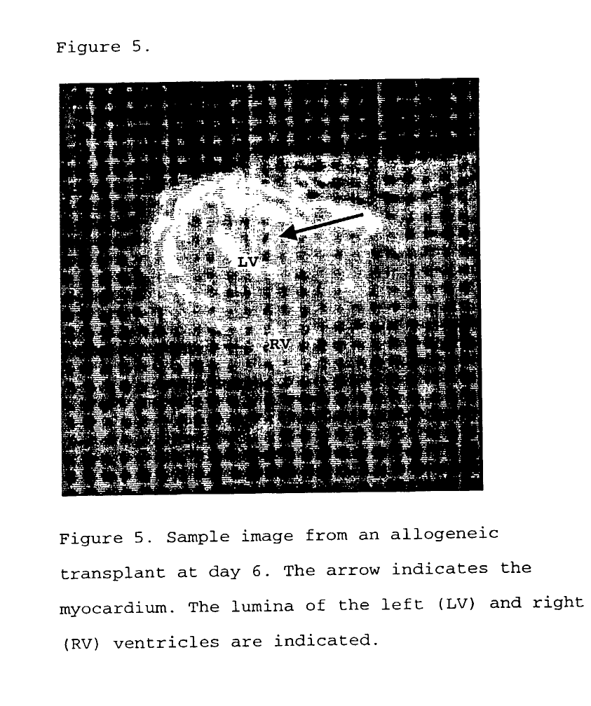

A image of an allogeneic transplant at day 6 is shown in FIG. 5.

1. A method of monitoring inter-endothelial discontinuities caused by an inflammatory disease in a human or vascularized animal subject, which comprises administering into the vasculature of the said subject a blood-pool MR contrast agent composition, and monitoring the MR signal for an increase in enhancement in said inter-endothelial discontinuities following administration of said composition.

2. A method as claimed in claim 1 of monitoring a transplanted organ in a human or vascularized animal subject, which comprises administering into the vasculature of the said subject into which an organ has been transplanted a blood-pool MR contrast agent composition, and monitoring the MR signal from the transplanted organ for an increase in enhancement following administration of said composition.

3. A method of detecting an indication of transplanted organ rejection which method comprises monitoring the MR signal from a transplanted organ in a vascularised human or non-human subject, following administration into the vasculature thereof of a blood-pool MR contrast agent composition, for a signal enhancement which is increased relative to that of the blood or of a non-transplanted vascularized tissue or organ, or which increases with time following the first pass of the agent through the transplant organ.

4. A method of generating enhanced images of a human or a vascularized animal subject previously administered with a blood-pool MR contrast agent composition, which method comprises generating an MR image of at least part of the inter-endothelial discontinuities of said body.

5. A method according to any of the previous claims wherein said blood-pool MR contrast agent composition comprises magnetic material optionally formulated with polymeric chelates, liposomes, carbohydrates, proteins, cascade polymers or dendrimer polymers.

6. A method according to any of the previous claims wherein said blood-pool MR contrast agent composition comprises magnetic metal oxide, especially a particulate superparamagnetic metal oxide.

7. A method according to any of the previous claims wherein said metal oxide is an ultra small superparamagnetic iron oxide.

8. A method according to any of the previous claims wherein the contrast agent composition comprises NC100150.

9. The use of a blood-pool MR contrast agent for the manufacture of a contrast agent composition for use in a method of diagnosis which involves MR monitoring the status of any chronic and/or acute inflammatory disease in a human or vascularized animal subject.

10. The use as claimed in claim 9 for monitoring of a transplanted organ in a human or non-human animal subject to detect indications of organ rejection.

11. The use of a blood-pool contrast agent composition in MR imaging of at least part of a human or vascularized animal subject for monitoring inter-endothelial discontinuities caused by an inflammatory disease by monitoring the MR signal for an increase in enhancement in said inter-endothelial discontinuities following administration of said composition.

12. The use as claimed in claims 9 to 11 wherein said blood-pool MR contrast composition comprises an ultra small superparamagnetic iron oxide.
